(19) United States
(12) Patent Application Publication
(10) Pub. No.: US 2026/0047891 A1
(43) Pub. Date: Feb. 19, 2026

(54) PUNCTURE SUPPORT APPARATUS AND PUNCTURE SUPPORT PROGRAM

(71) Applicant: FUJIFILM Corporation, Tokyo (JP)

(72) Inventor: Yuko SATO, Tokyo (JP)

(73) Assignee: FUJIFILM Corporation, Tokyo (JP)

(21) Appl. No.: 19/296,902

(22) Filed: Aug. 11, 2025

(30) Foreign Application Priority Data
Aug. 13, 2024 (JP) 2024-135139

(51) Int. Cl. A61B 34/10; A61B 18/00; A61B 90/00

(52) U.S. Cl. CPC A61B 34/10; A61B 18/00; A61B 90/361; A61B 90/39; A61B 2018/00577; A61B 2034/105; A61B 2034/107; A61B 2090/378; A61B 2090/3937

(57) ABSTRACT

A volume data acquisition unit acquires volume data including a puncture target object and an avoidance tissue. A model forming unit forms a subject model that is a three-dimensional model including a puncture target object model and an avoidance tissue model, based on the volume data. A puncture route specifying unit specifies, in the subject model, a recommended puncture route which is a route that extends from a body surface position of a subject E to the puncture target object model while avoiding the avoidance tissue model and through which a puncture needle is to pass. A display control unit notifies an operator of the specified recommended puncture route.

### CROSS REFERENCE TO RELATED APPLICATION

This application claims priority to Japanese Patent Application No. 2024-135139 filed on Aug. 13, 2024 which is incorporated herein by reference in its entirety including the specification, claims, drawings, and abstract.

### BACKGROUND OF THE INVENTION

### 1. Field of the Invention

The present specification discloses improvements in a puncture support apparatus and a puncture support program.

### 2. Description of the Related Art

An ultrasound diagnostic apparatus is known that transmits ultrasound from an ultrasound probe toward a subject, receives a reflected wave from the subject in the ultrasound probe, and performs various types of processing such as forming an ultrasound tomographic image representing a cross section in the subject, based on a reception signal formed from the reflected wave.

In addition, in the related art, for the purpose of collecting a tissue (for example, a tumor) of a subject, a puncture needle is inserted (punctured) into the tissue of the subject. An ultrasound probe comprising a puncture needle has also been proposed. With such an ultrasound probe, it is possible to perform puncture on a subject while transmitting ultrasound to the subject to form and display an ultrasound tomographic image.

JP1998-005223A (JP-H10-005223A) discloses a puncture support system comprising: a puncture needle; a probe guide that holds the puncture needle such that a deflection angle of the puncture needle can be set and that has a built-in ultrasound sensor capable of performing sector scanning around an axis of the puncture needle; a CCD camera that captures a marker attached to the puncture needle and a marker attached to the probe guide; an ultrasound diagnostic apparatus that forms an ultrasound tomographic image based on a signal from the ultrasound sensor built in the probe guide; and a monitor image constructing unit that calculates and displays on a display unit a relationship between a puncture depth of the puncture needle obtained based on the marker attached to the puncture needle in an image captured by the CCD camera, a deflection angle of the puncture needle obtained based on the marker attached to the probe guide in the image captured by the CCD camera, and a position of an internal target site (for example, a tumor) obtained from the ultrasound tomographic image.

### SUMMARY OF THE INVENTION

In a case of puncturing a puncture target object in a subject, it may be necessary to puncture the puncture target object while avoiding a tissue that is located around the puncture target object and that is to be avoided from being punctured (in the present specification, such a tissue is referred to as an “avoidance tissue”). The avoidance tissue is, for example, a tissue that affects the prognosis of the subject in a case of puncturing the avoidance tissue. Examples of the avoidance tissue include a blood vessel and a nerve.

As described above, although puncture has been performed while displaying the ultrasound tomographic image representing the cross section in the subject in the related art, it is still necessary for an operator such as a doctor to have experience and manual skills in order to perform puncture on the puncture target object while avoiding the avoidance tissue.

An object of the puncture support apparatus disclosed in the present specification is to provide an operator with a recommended puncture route which is a route that extends from a body surface of a subject to a puncture target object while avoiding an avoidance tissue that is to be avoided by a puncture needle and through which the puncture needle is to pass.

A puncture support apparatus disclosed in the present specification comprises: a probe information acquisition unit that acquires probe information indicating a position and a posture of an ultrasound probe; a volume data acquisition unit that acquires volume data based on a reception signal obtained by transmitting and receiving ultrasound from the ultrasound probe toward a subject; a model forming unit that forms a subject model including a puncture target object model that is a three-dimensional model representing a puncture target object, and an avoidance tissue model that is a three-dimensional model representing an avoidance tissue which is a tissue to be avoided by a puncture needle while puncturing the puncture target object, based on the volume data, the subject model having position information indicating each position of the subject model based on the position and the posture of the ultrasound probe in a case where the reception signal is acquired; a puncture route specifying unit that specifies a recommended puncture route which is a route that extends from a body surface position of the subject to the puncture target object model while avoiding the avoidance tissue model in the subject model and through which the puncture needle is to pass; and a notification processing unit that notifies an operator of the recommended puncture route.

The puncture route specifying unit may specify the recommended puncture route such that a shortest distance between the recommended puncture route and the avoidance tissue model is equal to or greater than a first threshold distance.

The puncture route specifying unit may specify, in a case where the puncture route specifying unit specifies a plurality of candidate puncture routes that extend from the body surface position of the subject to the puncture target object model while avoiding the avoidance tissue model, as the recommended puncture route, a candidate puncture route having a shortest length among the plurality of candidate puncture routes.

The puncture target object may be a tumor, the puncture needle may be an ablation puncture needle for ablating the tumor, and the puncture route specifying unit may specify the recommended puncture route such that an end part of the recommended puncture route on a tumor model side as the puncture target object model is at a second threshold distance or more from a tissue model other than the tumor model.

The ultrasound diagnostic apparatus may further comprise: a recommended position and posture specifying unit that specifies a recommended probe position and posture that corresponds to the position and the posture of the ultrasound probe such that a scanning plane of the ultrasound probe includes the recommended puncture route and is parallel to the recommended puncture route, based on the recommended puncture route, in which the notification processing unit notifies the operator of the recommended probe position and posture.

The notification processing unit may notify the operator of guide information for transitioning the position and the posture of the ultrasound probe from a current position and a current posture of the ultrasound probe to the recommended probe position and posture based on a difference between the current position and the current posture of the ultrasound probe and the recommended probe position and posture.

The ultrasound diagnostic apparatus may further comprise: a consistency index calculation unit that calculates a consistency index that is an index indicating a degree of matching between a reconstructed ultrasound image cut out from the subject model in a cross section that includes the recommended puncture route and is parallel to the recommended puncture route, and a real-time image that is an ultrasound tomographic image formed at the current position and the current posture of the ultrasound probe, in which the notification processing unit notifies the operator of the consistency index.

The ultrasound diagnostic apparatus may further comprise: a puncture needle information acquisition unit that acquires puncture needle information indicating a current position and a current posture of the puncture needle; and a puncture needle deviation determination unit that determines that the puncture needle has deviated from the recommended puncture route in a case where a distance between the puncture needle at the current position and the recommended puncture route becomes equal to or greater than a distance threshold or in a case where a difference in angle between a current extension direction of the puncture needle and an extension direction of the recommended puncture route becomes equal to or greater than an angle threshold, in which the notification processing unit notifies the operator in a case where it is determined that the puncture needle has deviated from the recommended puncture route.

The probe information acquisition unit may detect a position and a posture of the ultrasound probe relative to a body surface of the subject based on a captured image acquired by imaging a probe detection marker attached to the ultrasound probe and a body surface detection marker attached to the body surface of the subject via a camera.

The probe information acquisition unit may detect a position and a posture of the ultrasound probe relative to a body surface of the subject based on a captured image acquired by imaging a probe detection marker attached to the ultrasound probe and a body surface detection marker attached to the body surface of the subject via a camera, and the puncture needle information acquisition unit may detect a position and a posture of the puncture needle relative to the body surface of the subject based on a captured image acquired by imaging a puncture needle detection marker attached to the puncture needle and the body surface detection marker via the camera.

In addition, a puncture support program disclosed in the present specification causes a computer to function as: a probe information acquisition unit that acquires probe information indicating a position and a posture of an ultrasound probe; a volume data acquisition unit that acquires volume data based on a reception signal obtained by transmitting and receiving ultrasound from the ultrasound probe toward a subject; a model forming unit that forms a subject model including a puncture target object model that is a three-dimensional model representing a puncture target object, and an avoidance tissue model that is a three-dimensional model representing an avoidance tissue which is a tissue to be avoided by a puncture needle while puncturing the puncture target object, based on the volume data, the subject model having position information indicating each position of the subject model based on the position and the posture of the ultrasound probe in a case where the reception signal is acquired; and a puncture route specifying unit that specifies a recommended puncture route which is a route that extends from a body surface position of the subject to the puncture target object model while avoiding the avoidance tissue model in the subject model and through which the puncture needle is to pass.

With the puncture support apparatus disclosed in the present specification, it is possible to provide an operator with a recommended puncture route which is a route that extends from a body surface of a subject to a puncture target object while avoiding an avoidance tissue that is to be avoided by a puncture needle and through which the puncture needle is to pass.

### BRIEF DESCRIPTION OF THE DRAWINGS

FIG. 1 is a schematic diagram of a configuration of a puncture support system according to the present embodiment.

FIG. 2 is a diagram showing an example of an image captured by a camera.

FIG. 3 is a schematic diagram of a configuration of an ultrasound diagnostic apparatus according to the present embodiment.

FIG. 4 is a conceptual diagram showing a concept of processing of forming volume data.

FIG. 5 is a conceptual diagram showing a concept of processing of forming a subject model.

FIG. 6 is a diagram showing an example of the subject model.

FIG. 7 is a diagram showing an example of a recommended puncture route.

FIG. 8 is a diagram showing a display example of a reconstructed ultrasound image and a recommended puncture route image.

FIG. 9 is a first diagram showing a notification example of a recommended probe position and posture.

FIG. 10 is a second diagram showing the notification example of the recommended probe position and posture.

FIG. 11 is a diagram showing a display example of a real-time image having the same cross section as the reconstructed ultrasound image.

FIG. 12 is a diagram showing a current puncture needle and a recommended puncture route in a model coordinate system.

FIG. 13 is a diagram showing an example of notification that a puncture needle has deviated.

FIG. 14 is a diagram showing an example of notification that the puncture needle has not deviated.

### DESCRIPTION OF THE PREFERRED EMBODIMENTS

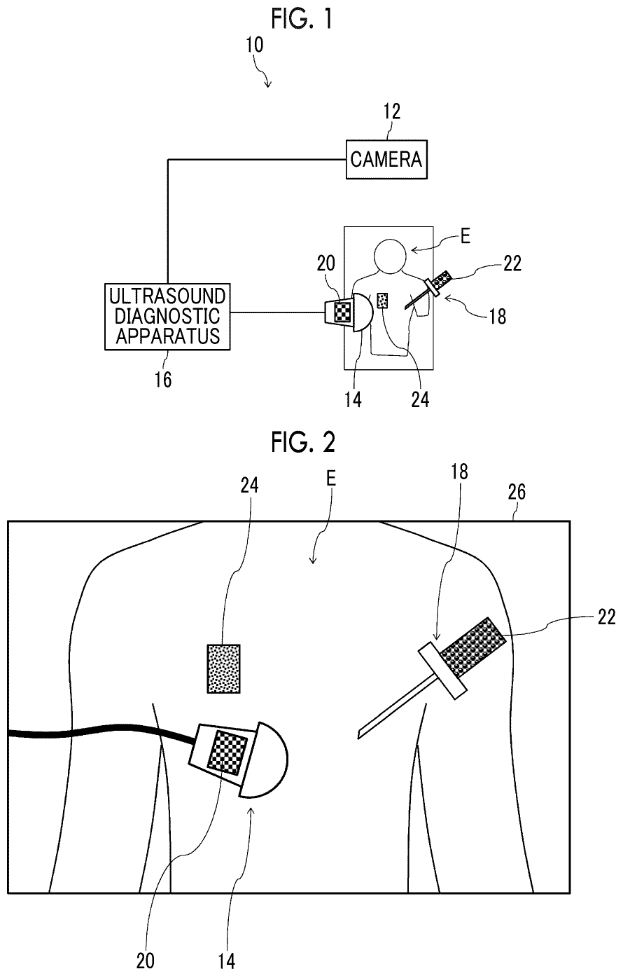

FIG. 1 is a schematic diagram of a configuration of a puncture support system 10 according to the present embodiment. The puncture support system 10 is configured to include a camera 12, an ultrasound diagnostic apparatus 16 as a puncture support apparatus including an ultrasound probe 14, and a puncture needle 18. In the present embodiment, the ultrasound probe 14 and the puncture needle 18 are separate bodies, but the ultrasound probe 14 may be a puncture probe, that is, the puncture needle 18 may be provided in the ultrasound probe 14.

The camera 12 and the ultrasound diagnostic apparatus 16 are communicatively connected to each other.

In the present embodiment, a probe detection marker 20 is attached to the ultrasound probe 14. The probe detection marker 20 is a marker for detecting a position and a posture of the ultrasound probe 14. In addition, a puncture needle detection marker 22 is attached to the puncture needle 18. The puncture needle detection marker 22 has a pattern different from the probe detection marker 20 and is a marker for detecting a position and a posture of the puncture needle 18. Further, a body surface detection marker 24 is attached to a body surface of a subject E. The body surface detection marker 24 has a different pattern from the probe detection marker 20 and the puncture needle detection marker 22 and is a marker for detecting a position and a posture of the body surface of the subject E. An example of the probe detection marker 20, the puncture needle detection marker 22, and the body surface detection marker 24 is an augmented reality (AR) marker.

The camera 12 is configured to include, in addition to a lens and an image sensor, a processor including a central processing unit (CPU) and the like, a communication interface including a network adapter and the like, and the like. The camera 12 images the ultrasound probe 14 (specifically, the probe detection marker 20), the puncture needle 18 (specifically, the puncture needle detection marker 22), and the subject E (specifically, the body surface detection marker 24). A captured image is formed by the image sensor of the camera 12, and the captured image is transmitted to the ultrasound diagnostic apparatus 16 via the communication interface of the camera 12.

FIG. 2 is a diagram showing an example of a captured image 26 of the camera 12. As described above, the captured image 26 includes the images of the probe detection marker 20, the puncture needle detection marker 22, and the body surface detection marker 24. The ultrasound diagnostic apparatus 16 can detect the position and the posture of the ultrasound probe 14 by analyzing the image of the probe detection marker 20 shown in the captured image 26. In addition, the ultrasound diagnostic apparatus 16 can detect the position and the posture of the puncture needle 18 by analyzing the image of the puncture needle detection marker 22 shown in the captured image 26. Further, the ultrasound diagnostic apparatus 16 can detect the position and the posture of the body surface of the subject E by analyzing the image of the body surface detection marker 24 shown in the captured image 26. Details of processing of detecting the positions and postures of the ultrasound probe 14, the puncture needle 18, and the subject E will be described below.

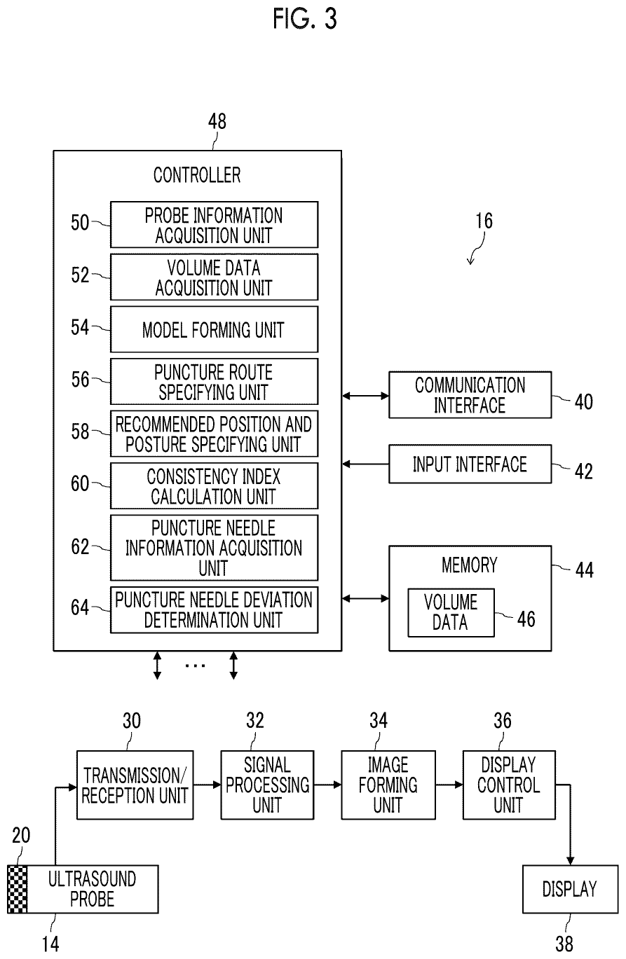

FIG. 3 is a schematic diagram of a configuration of the ultrasound diagnostic apparatus 16. The ultrasound diagnostic apparatus 16 is a medical apparatus installed in medical institutions such as a hospital.

The ultrasound probe 14 is a device that transmits and receives ultrasound to and from the subject E. The ultrasound probe 14 has a transducer element array including a plurality of transducer elements. In the present embodiment, the transducer element array is formed of the plurality of transducer elements arranged in one direction (array direction). In a case where a transmission signal is supplied to each transducer element from a transmission/reception unit 30, which will be described later, each transducer element generates ultrasound. Specifically, the ultrasound probe 14 scans an ultrasound beam on a plane (scanning plane) parallel to the array direction.

As described above, the probe detection marker 20 is attached to the ultrasound probe 14.

The transmission/reception unit 30 transmits a transmission signal to the ultrasound probe 14 (specifically, each transducer element of the transducer element array) under control of a controller 48, which will be described later. As a result, the ultrasound beam is scanned on the scanning plane. In addition, the transmission/reception unit 30 receives a reception signal from each transducer element that has received a reflected wave from a target tissue.

The transmission/reception unit 30 includes an adder and a plurality of delay devices corresponding to the respective transducer elements and performs phase alignment and addition processing of aligning and adding phases of the reception signals from the transducer elements by using the adder and the plurality of delay devices. As a result, a reception beam signal in which information indicating a signal intensity of the reflected wave from the target tissue is arranged in a depth direction of the target tissue is formed.

A signal processing unit 32 performs various types of signal processing including filter processing of applying a bandpass filter, detection processing, and the like, on the reception beam signal from the transmission/reception unit 30.

An image forming unit 34 forms an ultrasound tomographic image (B-mode image) representing a cross section (particularly, the scanning plane of the ultrasound beam) of the subject E based on the reception beam signal subjected to the signal processing in the signal processing unit 32.

A display control unit 36 performs control to display various images including the ultrasound tomographic image formed by the image forming unit 34 on a display 38.

The display 38 as a display unit is, for example, a display device configured using a liquid-crystal display, an organic electroluminescence (EL), or the like.

The transmission/reception unit 30, the signal processing unit 32, the image forming unit 34, and the display control unit 36 included in the ultrasound diagnostic apparatus 16 are configured by a processor. The processor is configured to include at least one of a general-purpose processing device (for example, a CPU or the like) or a dedicated processing device (for example, a graphics processing unit (GPU), an application-specific integrated circuit (ASIC), a field-programmable gate array (FPGA), or a programmable logic device). The processor need not be configured using a single processing device but rather may be configured through cooperation of a plurality of processing devices that are present at physically separated positions. Additionally, each of the above-described units may be implemented through cooperation of hardware, such as a processor, and software.

A communication interface 40 is configured using, for example, a network adapter. The communication interface 40 exhibits a function of communicating with another device (particularly, the camera 12). In particular, the communication interface 40 receives the captured image 26 from the camera 12.

An input interface 42 is configured using, for example, a button, a trackball, or a touch panel. The input interface 42 is used to input a command of an operator, such as a doctor, who uses the ultrasound diagnostic apparatus 16, to the ultrasound diagnostic apparatus 16.

Amemory 44 is configured to include a hard disk drive (HDD), a solid-state drive (SSD), an embedded Multi Media Card (eMMC), a read-only memory (ROM), a random-access memory (RAM), or the like. A puncture support program for operating each unit of the ultrasound diagnostic apparatus 16 is stored in the memory 44. In addition, the puncture support program can also be stored in a non-transitory computer-readable storage medium such as a Universal Serial Bus (USB) memory or a CD-ROM. The ultrasound diagnostic apparatus 16 can read and execute the puncture support program from such a storage medium. Since the ultrasound diagnostic apparatus 16 reads the puncture support program to exhibit the functions described below, the ultrasound diagnostic apparatus 16 can be said to be a computer program product.

As shown in FIG. 3, volume data 46 is stored in the memory 44. In the present embodiment, the volume data 46 is formed based on a plurality of ultrasound tomographic images based on the reception signals acquired by the operator while moving the ultrasound probe 14 in a direction perpendicular to the scanning plane. Details of the volume data 46 will be described later.

The controller 48 includes at least one of a general-purpose processor (such as a CPU) or a dedicated processor (such as a GPU, an ASIC, an FPGA, or a programmable logic device). The controller 48 need not be configured using a single processing device but rather may be configured through cooperation of a plurality of processing devices that are present at physically separated positions. The controller 48 controls each unit of the ultrasound diagnostic apparatus 16. In addition, as shown in FIG. 3, the controller 48 exhibits functions as a probe information acquisition unit 50, a volume data acquisition unit 52, a model forming unit 54, a puncture route specifying unit 56, a recommended position and posture specifying unit 58, a consistency index calculation unit 60, a puncture needle information acquisition unit 62, and a puncture needle deviation determination unit 64 in accordance with the puncture support program stored in the memory 44.

The probe information acquisition unit 50 acquires probe information indicating the position and the posture of the ultrasound probe 14.

In the present embodiment, the probe information acquisition unit 50 acquires the probe information by analyzing the captured image 26 acquired by the camera 12 to detect the position and the posture of the ultrasound probe 14. As described above, the captured image 26 includes the image of the probe detection marker 20 for detecting the position and the posture of the ultrasound probe 14 (see FIG. 2). The probe information acquisition unit 50 acquires the probe information by analyzing the image of the probe detection marker 20 in the captured image 26. The probe information includes position information indicating the position of the ultrasound probe 14 and posture information indicating the posture of the ultrasound probe 14.

The position information may be, for example, three-dimensional coordinates in a camera coordinate system. The posture information may be a rotation angle with respect to a predetermined axis (for example, an X axis, a Y axis, or a Z axis) in the camera coordinate system. Since a known method can be used as a method of detecting the position and the posture of the ultrasound probe 14 in the camera coordinate system from the image of the probe detection marker 20 included in the captured image 26, detailed descriptions thereof will be omitted.

The probe information acquisition unit 50 may perform calibration prior to detecting the position and the posture of the ultrasound probe 14 based on the captured image 26.

Specifically, the operator sets the position and the posture of the ultrasound probe 14 to a predetermined position and posture, and inputs a calibration instruction to the ultrasound diagnostic apparatus 16 in that state. The probe information acquisition unit 50 detects the position and the posture of the ultrasound probe 14 in a case where the calibration instruction is input, based on the probe detection marker 20 included in the captured image 26, and sets the position and the posture as a reference position and a reference posture. As a result, it is possible to obtain a relationship between the information (for example, the position and the posture in the camera coordinate system) indicating the position and the posture of the ultrasound probe 14 detected by the probe information acquisition unit 50 and a real space coordinate system.

As described above, the captured image 26 also includes the image of the body surface detection marker 24 for detecting the position and the posture of the body surface of the subject E (see FIG. 2). The probe information acquisition unit 50 may detect the position and the posture of the body surface of the subject E by analyzing the image of the body surface detection marker 24 in the captured image 26. In addition, the probe information acquisition unit 50 may detect the position and the posture of the ultrasound probe 14 relative to the position and the posture of the body surface of the subject E. As a result, the position and the posture of the ultrasound probe 14 with respect to the subject E, which absorb any variation in the position or the posture of the subject E, can be obtained.

The probe information acquisition unit 50 may detect the position and the posture of the ultrasound probe 14 by a method other than analyzing the captured image 26. For example, a position and posture sensor such as a magnetic sensor may be provided in the ultrasound probe 14, and the position and the posture of the ultrasound probe 14 may be detected based on a detection value of the position and posture sensor.

The scanning plane, which is a plane on which the ultrasound beam from the ultrasound probe 14 is scanned, is determined by the position and the posture of the ultrasound probe 14.

Therefore, it can be said that the probe information acquired by the probe information acquisition unit 50 indicates a position and a posture of the scanning plane.

The volume data acquisition unit 52 acquires the volume data 46 based on the reception signal obtained by transmitting and receiving ultrasound from the ultrasound probe 14 toward the subject E. In the present embodiment, the volume data acquisition unit 52 forms the volume data 46 based on the reception signals corresponding to a plurality of the scanning planes acquired by the operator while moving the ultrasound probe 14 in a direction perpendicular to the scanning plane.

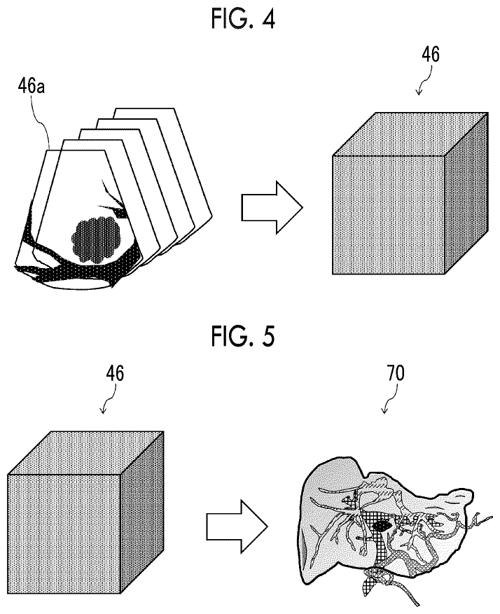

FIG. 4 is a conceptual diagram showing a concept of processing of forming the volume data 46. In the present embodiment, first, the operator performs transmission and reception of ultrasound to and from the subject E while moving the ultrasound probe 14 in a direction perpendicular to the scanning plane. Here, it is assumed that a puncture target object, which is a puncture target, inside the subject E and an avoidance tissue around the puncture target object are included in a movement path of the scanning plane. As a result, the transmission/reception unit 30 acquires a plurality of the reception signals for the plurality of scanning planes arranged in the direction perpendicular to the scanning plane. Then, the image forming unit 34 forms a plurality of ultrasound tomographic images 46a corresponding to the plurality of scanning planes based on the plurality of reception signals. At least some of the plurality of ultrasound tomographic images 46a include an image of the puncture target object or an image of the avoidance tissue.

The probe information acquisition unit 50 detects the position and the posture of the ultrasound probe 14 in a case where each reception signal corresponding to each ultrasound tomographic image 46a is acquired, and associates the probe information indicating the position and the posture of the ultrasound probe 14 detected by the probe information acquisition unit 50 with each ultrasound tomographic image 46a. As described above, the probe information may indicate the position and the posture of the ultrasound probe 14 relative to the subject E. Each position (coordinate) on the ultrasound tomographic image 46a can be specified based on the position and the posture of the ultrasound probe 14 in a case where the reception signal corresponding to the ultrasound tomographic image 46a is acquired. That is, it can be said that each ultrasound tomographic image 46a includes position information indicating each position (coordinate) of the ultrasound tomographic image 46a based on the position and the posture of the ultrasound probe 14 in a case where the reception signal corresponding to the ultrasound tomographic image 46a is acquired.

The volume data acquisition unit 52 forms the volume data 46 based on the plurality of ultrasound tomographic images 46a as the reception signals. Since a known method can be used as a method of forming the volume data 46 from the plurality of ultrasound tomographic images 46a, detailed descriptions thereof will be omitted here. As described above, since the puncture target object and the avoidance tissue are included in the movement path of the scanning plane, and the image of the puncture target object or the image of the avoidance tissue is included in at least some of the plurality of ultrasound tomographic images 46a, the volume data 46 is data including the puncture target object and the avoidance tissue. In addition, as described above, since each ultrasound tomographic image 46a includes coordinate information indicating each position of the ultrasound tomographic image 46a based on the position and the posture of the ultrasound probe 14 in a case where the reception signal corresponding to the ultrasound tomographic image 46a is acquired, the volume data 46 made up of the plurality of ultrasound tomographic images 46a also has coordinate information indicating each position of the volume data 46 based on the position and the posture of the ultrasound probe 14 in a case where the reception signal corresponding to each ultrasound tomographic image 46a is acquired (referred to as a model coordinate system in the present specification).

In the present embodiment, the volume data 46 is made up of the plurality of ultrasound tomographic images 46a. However, in a case where a 2D array probe in which the transducer elements are two-dimensionally arranged is used as the ultrasound probe 14, the volume data acquisition unit 52 may directly form the volume data 46 including the puncture target object and the avoidance tissue based on two-dimensional reception signals from the ultrasound probe 14. Even in this case, the volume data 46 can be specified based on the position and the posture of the ultrasound probe 14 in a case where the volume data 46 is formed. That is, even in this case, the volume data 46 has coordinate information indicating each position of the volume data 46 in the model coordinate system.

The model forming unit 54 forms a subject model that is a three-dimensional model, including a puncture target object model that is a three-dimensional model representing the puncture target object, and an avoidance tissue model that is a three-dimensional model representing the avoidance tissue, based on the volume data 46.

FIG. 5 is a conceptual diagram showing a concept of processing of forming a subject model 70. Since a known method can be used as a method of forming the subject model 70 based on the volume data 46, detailed descriptions thereof will be omitted here, but the model forming unit 54 forms the subject model 70 using a technology such as volume rendering or surface rendering.

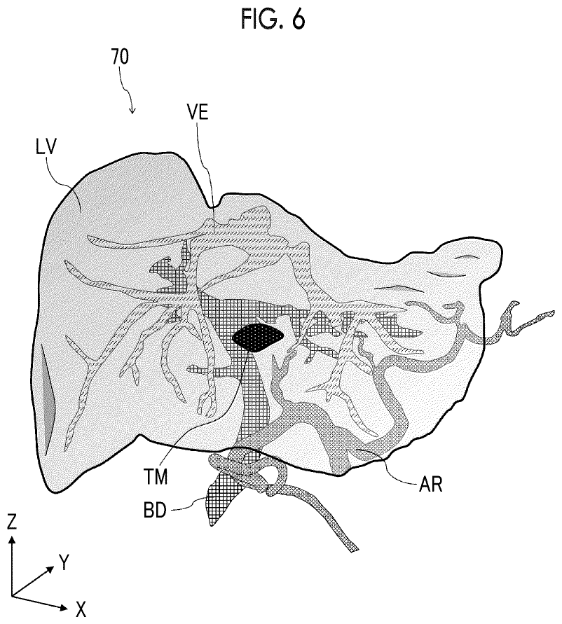

FIG. 6 is a diagram showing an example of the subject model 70. In the example of FIG. 6, the subject model 70 is a model representing a liver of the subject E and a periphery thereof. The subject model 70 is configured to include a plurality of tissue models. In the example of FIG. 6, the subject model 70 includes a liver model LV, a vein model VE, an artery model AR, a bile duct model BD, and a tumor model TM. Among these, in the present embodiment, since a tumor in the liver is the puncture target object, the tumor model TM is the puncture target object model, and since a vein, an artery, and a bile duct in the liver are the avoidance tissues, the vein model VE, the artery model AR, and the bile duct model BD are the avoidance tissue models. It should be noted that, strictly speaking, the subject model 70 representing the periphery of the liver also includes models of an inferior vena cava, a portal vein, vascular regions, and other tissues; but these are not shown in FIG. 6.

Since the volume data 46 has the position information in the model coordinate system, and the subject model 70 is formed from the volume data 46, the subject model 70 also has the position information indicating each position of the subject model 70 based on the position and the posture of the ultrasound probe 14 in a case where the reception signals constituting the volume data 46 are acquired (in the model coordinate system). Since the position of the ultrasound probe 14 in a case where the reception signal is acquired represents a body surface position of the subject E, the body surface position of the subject E in the subject model 70 is also known.

FIG. 6 shows three-axis directions (X-axis, Y-axis, and Z-axis) of the model coordinate system indicating the position of the subject model 70. In a case where the position and the posture of the ultrasound probe 14 are acquired by analyzing the captured image 26 acquired by the camera 12, and the position and an orientation of the camera 12 are fixed, the model coordinate system may be the same as the camera coordinate system of the camera 12. In addition, a relationship between the model coordinate system and the real space coordinate system can be obtained by the above-described calibration.

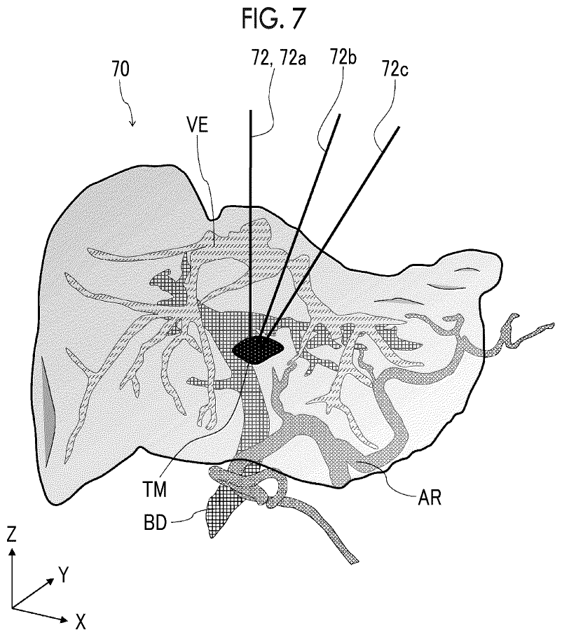

The puncture route specifying unit 56 specifies a recommended puncture route that is a route through which the puncture needle 18 is to pass, in the subject model 70. FIG. 7 is a diagram showing an example of a recommended puncture route 72. In particular, the puncture route specifying unit 56 specifies a recommended puncture route 72 that extends from the body surface position of the subject E to the puncture target object model (in the example of FIG. 7, the tumor model TM) while avoiding the avoidance tissue models (in the example of FIG. 7, the vein model VE, the artery model AR, and the bile duct model BD).

Specifically, the puncture route specifying unit 56 searches for a straight line in the subject model 70 that extends from the puncture target object model toward the body surface position of the subject E (the body surface position is not limited to a single point) and that does not pass through the avoidance tissue models. In a case where such a straight line is found, the straight line is specified as a candidate for the recommended puncture route 72 (referred to as a “candidate puncture route” in the present specification). In particular, the puncture route specifying unit 56 may specify the candidate puncture route such that a shortest distance between the candidate puncture route and the avoidance tissue model is equal to or greater than a predetermined first threshold distance. This is to reduce a likelihood that the puncture needle 18 punctures the avoidance tissue model in a case where the operator actually performs puncture along the recommended puncture route 72 later.

In a case where only one candidate puncture route is found, the puncture route specifying unit 56 may specify the candidate puncture route as the recommended puncture route 72.

However, typically, a plurality of candidate puncture routes 72a to 72c are specified as shown in FIG. 7. In this case, the puncture route specifying unit 56 specifies, as the recommended puncture route 72, a candidate puncture route having the shortest length (for example, the candidate puncture route 72a) among the plurality of candidate puncture routes 72a to 72c. This is because, in a case where the operator actually performs puncture along the recommended puncture route 72 later, a shorter puncture route is less likely to result in an unexpected event (such as a mistake by the operator).

In addition, in a case where the puncture target object is a tumor, the puncture needle 18 may be an ablation puncture needle for ablating the tumor. The ablation puncture needle ablates the tumor by delivering a high-frequency radio wave current to the tumor from a tip of the puncture needle 18. In this case, in a case where there is a tissue other than the tumor in the vicinity of the tip of the puncture needle 18, the surrounding tissue may be affected during the ablation of the tumor. In consideration of this, in a case where the puncture target object is a tumor and the puncture needle 18 is an ablation puncture needle, the puncture route specifying unit 56 may specify the recommended puncture route 72 such that an end part of the recommended puncture route 72 on a tumor model TM side as the puncture target object model is at a predetermined second threshold distance or more from the tissue model other than the tumor model TM.

The display control unit 36 as a notification processing unit notifies the operator of the recommended puncture route 72 specified by the puncture route specifying unit 56. In the present embodiment, the display control unit 36 notifies the operator of the recommended puncture route 72 by a method described below.

First, the image forming unit 34 specifies, within the volume data 46, a cross section that includes the recommended puncture route 72 and that is parallel to the recommended puncture route 72. As described above, since the volume data 46 and the subject model 70 have the position information in the same model coordinate system, the image forming unit 34 can specify the cross section that includes the recommended puncture route 72 and that is parallel to the recommended puncture route 72 within the volume data 46 based on the recommended puncture route 72 specified in the subject model 70.

The image forming unit 34 cuts out and reconstructs the volume data 46 in the specified cross section to form a reconstructed ultrasound image. Then, the display control unit 36 displays, together with the formed reconstructed ultrasound image, a recommended puncture route image indicating the recommended puncture route 72 superimposed on the reconstructed ultrasound image, on the display 38. As a result, the display control unit 36 notifies the operator of the recommended puncture route 72.

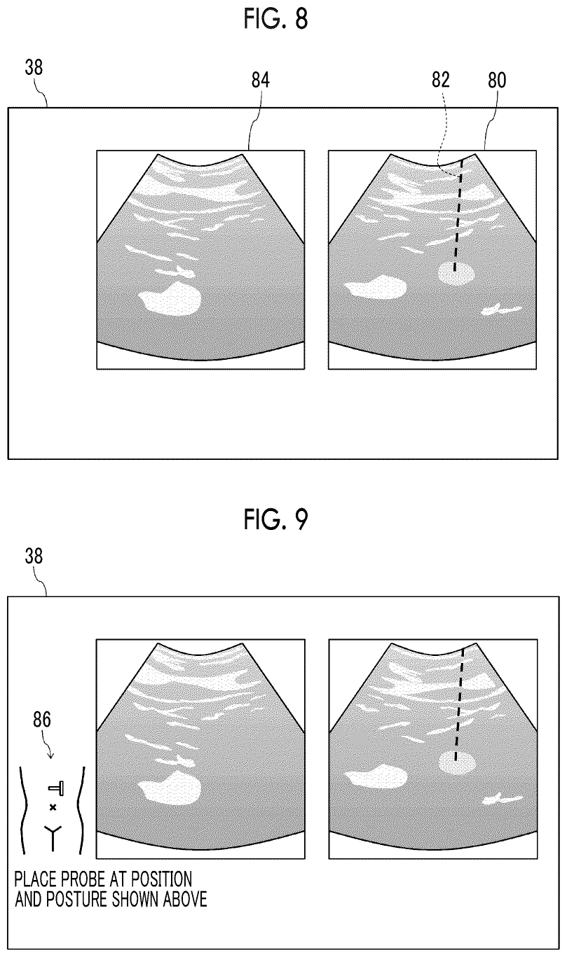

FIG. 8 is a diagram showing a display example of a reconstructed ultrasound image 80 and a recommended puncture route image 82. In the example of FIG. 8, the recommended puncture route image 82 is indicated by a dashed line.

A method of notifying the recommended puncture route 72 by the display control unit 36 is not limited to the above method. For example, the display control unit 36 may notify the operator of the recommended puncture route 72 by displaying the three-dimensional subject model 70 in which the recommended puncture route 72 is shown on the display 38.

As described above, according to the present embodiment, the puncture route specifying unit 56 specifies the recommended puncture route 72 that extends from the body surface position of the subject E to the puncture target object while avoiding the avoidance tissue, and the operator is notified of the recommended puncture route 72. As a result, the operator can easily ascertain the puncture route for puncturing the puncture target object while avoiding the avoidance tissue.

As shown in FIG. 8, the display control unit 36 may display a real-time image 84 that is an ultrasound tomographic image formed at the current position and posture of the ultrasound probe 14, together with the reconstructed ultrasound image 80 and the recommended puncture route image 82. In a case where the puncture needle 18 is inserted into the subject E, and the puncture needle 18 passes through the scanning plane of the ultrasound probe 14 (particularly, in a case where the puncture needle 18 and the scanning plane are parallel to each other), an image of the puncture needle 18 appears in the real-time image 84. Therefore, in a case where the operator regards a cross section of the real-time image 84 as the same cross section as that of the reconstructed ultrasound image 80, the operator can perform puncture along the recommended puncture route 72 while comparing the recommended puncture route image 82 on the reconstructed ultrasound image 80 with the image of the puncture needle 18 appearing in the real-time image 84.

The recommended position and posture specifying unit 58 specifies a recommended probe position and posture that corresponds to the position and the posture of the ultrasound probe 14 for forming the real-time image 84 of the same cross section as that of the reconstructed ultrasound image 80. As described above, the reconstructed ultrasound image 80 represents a cross section that includes the recommended puncture route 72 and that is parallel to the recommended puncture route 72 within the volume data 46. Therefore, the recommended position and posture specifying unit 58 also first specifies a cross section that includes the recommended puncture route 72 and that is parallel to the recommended puncture route 72 within the volume data 46 based on the recommended puncture route 72. Then, the body surface position included in the specified cross section is the position of the ultrasound probe 14 for forming the real-time image 84 of the same cross section as that of the reconstructed ultrasound image 80 in the model coordinate system, and the posture in which the specified cross section is parallel to the scanning plane is the posture of the ultrasound probe 14 for forming the real-time image 84 of the same cross section as that of the reconstructed ultrasound image 80 in the model coordinate system. Further, the recommended position and posture specifying unit 58 converts the recommended probe position and posture specified in the model coordinate system into a recommended probe position and posture in the real space based on the relationship between the model coordinate system and the real space coordinate system. The recommended probe position and posture obtained in this way corresponds to a position and a posture in which the scanning plane of the ultrasound probe 14 includes the recommended puncture route 72 (in the real space) and is parallel to the recommended puncture route 72.

The display control unit 36 as the notification processing unit notifies the operator of the recommended probe position and posture specified by the recommended position and posture specifying unit 58.

FIG. 9 is a first diagram showing a notification example of the recommended probe position and posture. For example, as shown in FIG. 9, the display control unit 36 displays a body mark 86 indicating an outline of the subject E on the display 38 and indicates the recommended probe position and posture on the body mark 86 to notify the operator of the recommended probe position and posture.

In addition, the probe information acquisition unit 50 may detect the current position and posture of the ultrasound probe 14, and the display control unit 36 may notify the operator of guide information for transitioning the position and posture of the ultrasound probe 14 from the current position and posture of the ultrasound probe 14 to the recommended probe position and posture based on a difference between the current position and posture of the ultrasound probe 14 and the recommended probe position and posture.

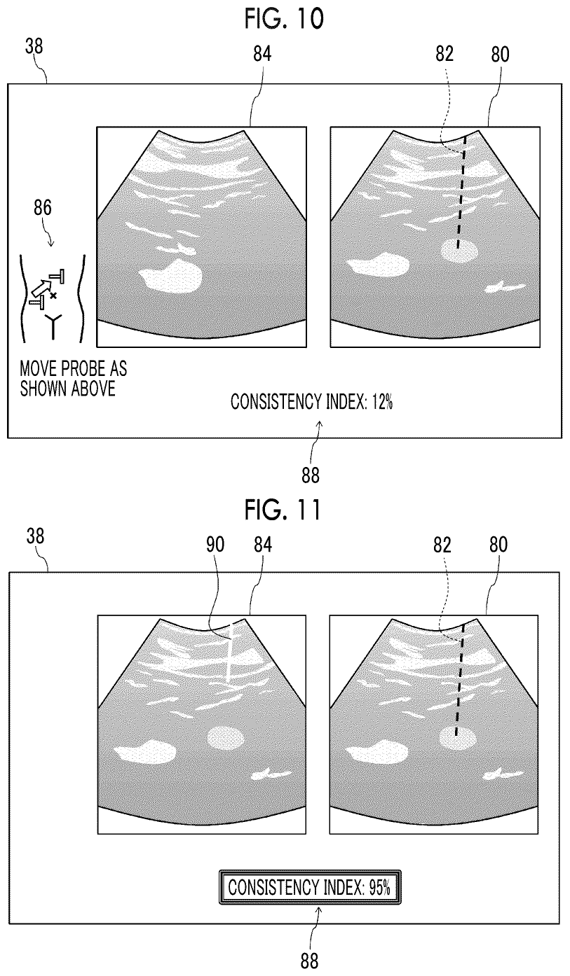

FIG. 10 is a second diagram showing the notification example of the recommended probe position and posture. For example, as shown in FIG. 10, the display control unit 36 may display the body mark 86 indicating the outline of the subject E on the display 38, and may display the current position and posture of the ultrasound probe 14 and the recommended probe position and posture as the guide information on the body mark 86. Such guide information may be a moving image showing the transition from the current position and posture of the ultrasound probe 14 to the recommended probe position and posture.

The notification aspect of the recommended probe position and posture is not limited to the above.

The operator adjusts the position and the posture of the ultrasound probe 14 such that the cross sections of the reconstructed ultrasound image 80 and the real-time image 84 are the same based on the recommended probe position and posture that was notified of.

The consistency index calculation unit 60 calculates a consistency index that is an index indicating a degree of matching between the reconstructed ultrasound image 80 and the real-time image 84. In the present embodiment, the consistency index calculation unit 60 calculates the consistency index by comprehensively evaluating a similarity between the reconstructed ultrasound image 80 and the real-time image 84, a difference between the position indicated by the recommended probe position and posture and the current position of the ultrasound probe 14, and a difference between the posture indicated by the recommended probe position and posture and the current posture of the ultrasound probe 14. For example, the consistency index calculation unit 60 calculates the consistency index according to Equation 1.

$$( Consistency index ) = ( Similarity between reconstructed ultrasound image 80 and real ‐ time image 84 ) \times {α}_{1} + ( Difference between position indicated by recommended probe position and posture and current position of ultrasound probe 14 ) \times {α}_{2} + ( Difference between posture indicated by recommended probe position and posture and current posture of ultrasound probe 14 ) \times {α}_{3} \qquad ( Equation 1 )$$

In Equation 1, a₁, a₂, and a₃ are coefficients (weights), and may be appropriately set in advance.

The display control unit 36 as the notification processing unit notifies the operator of the consistency index calculated by the consistency index calculation unit 60.

For example, as shown in FIG. 10, the display control unit 36 displays a consistency index image 88 representing the calculated consistency index on the display 38 together with the reconstructed ultrasound image 80 and the real-time image 84. It should be noted that the notification method of the consistency index is not limited thereto. For example, an audio output unit as the notification processing unit may notify the operator of the consistency index by voice.

FIG. 11 is a diagram showing a display example of the real-time image 84 which has the same cross section as that of the reconstructed ultrasound image 80. The operator can bring the cross section of the real-time image 84 closer to the cross section of the reconstructed ultrasound image 80 by appropriately adjusting the position and the posture of the ultrasound probe 14. In the example of FIG. 11, the consistency index reaches a fairly high value of 95%. The display control unit 36 may notify the operator that the consistency index is equal to or greater than a predetermined index threshold. For example, in a case where the consistency index is equal to or greater than the predetermined index threshold, the display control unit 36 may notify the operator that the consistency index is equal to or greater than the predetermined index threshold by displaying the consistency index image 88 in an emphasized manner.

In a case where the real-time image 84 of the same cross section as that of the reconstructed ultrasound image 80 can be visualized, the operator starts puncturing the puncture target object while referring to the recommended puncture route 72 and the real-time image 84. Here, the ultrasound diagnostic apparatus 16 according to the present embodiment assists the operator in performing puncture along the recommended puncture route 72.

The puncture needle information acquisition unit 62 acquires puncture needle information indicating the current position and posture of the puncture needle 18 in the model coordinate system.

In the present embodiment, the puncture needle information acquisition unit 62 acquires the puncture needle information by analyzing the captured image 26 (see FIG. 2) acquired by the camera 12 to detect the position and the posture of the puncture needle 18. As described above, the captured image 26 includes the image of the puncture needle detection marker 22 for detecting the position and the posture of the puncture needle 18. The puncture needle information acquisition unit 62 acquires the puncture needle information by analyzing the image of the puncture needle detection marker 22 in the captured image 26. The puncture needle information includes position information indicating the position of the puncture needle 18 and posture information indicating the posture of the puncture needle 18.

The position information may be, for example, three-dimensional coordinates in a camera coordinate system. As the position of the puncture needle 18, a position of a base of the puncture needle 18 (that is, a portion that is not inserted into the subject E) is detected. However, since a shape (for example, length) of the puncture needle 18 is known information, the position of the portion (for example, the tip of the puncture needle 18) of the puncture needle 18 inserted into the subject E can be detected based on the position information of the puncture needle 18 and the shape of the puncture needle 18.

In addition, the posture information may be a rotation angle with respect to a predetermined axis (for example, the X-axis, the Y-axis, or the Z-axis) in the camera coordinate system. The posture of the puncture needle 18 is, in other words, an extension direction of the puncture needle 18.

Since a known method can be used as a method of detecting the position and the posture of the puncture needle 18 in the camera coordinate system from the image of the puncture needle detection marker 22 included in the captured image 26, detailed descriptions thereof will be omitted.

The puncture needle information acquisition unit 62 may perform calibration prior to detecting the position and the posture of the puncture needle 18 based on the captured image 26. Specifically, the operator sets the position and the posture of the puncture needle 18 to a predetermined position and posture, and inputs a calibration instruction to the ultrasound diagnostic apparatus 16 in that state. The puncture needle information acquisition unit 62 detects the position and the posture of the puncture needle 18 in a case where the calibration instruction is input, based on the puncture needle detection marker 22 included in the captured image 26, and sets the position and the posture as a reference position and a reference posture. As a result, it is possible to obtain a relationship between the information (for example, the position and the posture in the camera coordinate system) indicating the position and the posture of the puncture needle 18 detected by the puncture needle information acquisition unit 62 and the real space coordinate system.

As described above, the captured image 26 also includes the image of the body surface detection marker 24 for detecting the position and the posture of the body surface of the subject E (see FIG. 2). The puncture needle information acquisition unit 62 may detect the position and the posture of the body surface of the subject E by analyzing the image of the body surface detection marker 24 in the captured image 26. In addition, the puncture needle information acquisition unit 62 may detect the position and the posture of the puncture needle 18 relative to the position and the posture of the body surface of the subject E. As a result, the position and the posture of the puncture needle 18 with respect to the subject E, which absorb any variation in the position or the posture of the subject E, can be obtained.

The puncture needle information acquisition unit 62 may detect the position and the posture of the puncture needle 18 by a method other than analyzing the captured image 26. For example, a position and posture sensor such as a magnetic sensor may be provided in the puncture needle 18, and the position and the posture of the puncture needle 18 may be detected based on a detection value of the position and posture sensor.

The puncture needle information acquisition unit 62 converts the puncture needle information in the real space coordinate system (or the camera coordinate system) into the puncture needle information in the model coordinate system based on the relationship between the real space coordinate system and the model coordinate system.

The puncture needle deviation determination unit 64 determines that the puncture needle 18 has deviated from the recommended puncture route 72 in a case where a distance between the puncture needle 18 at the current position and the recommended puncture route 72 in the model coordinate system is equal to or greater than a predetermined distance threshold, based on the puncture needle information acquired by the puncture needle information acquisition unit 62.

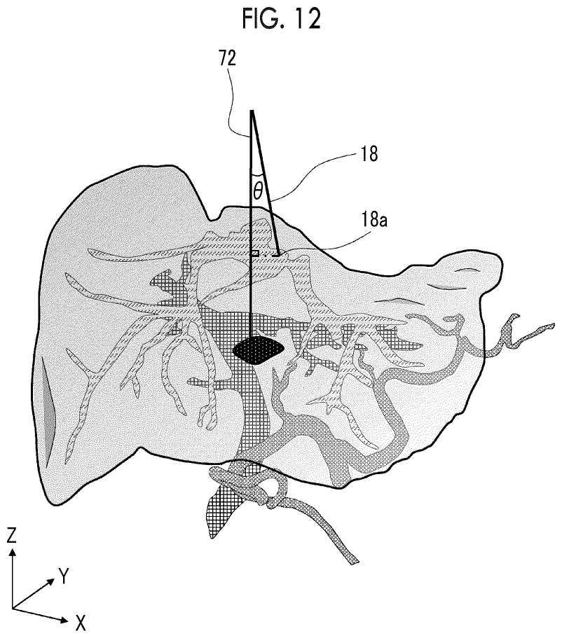

FIG. 12 is a diagram showing the current puncture needle 18 and the recommended puncture route 72 in the model coordinate system. Since both the puncture needle 18 and the recommended puncture route 72 have an extending shape, for example, the puncture needle deviation determination unit 64 may set a maximum distance between the puncture needle 18 and the recommended puncture route 72 (in the example of FIG. 12, a length of a perpendicular line drawn from a tip 18a of the puncture needle 18 to the recommended puncture route 72) as the distance between the current position of the puncture needle 18 and the recommended puncture route 72.

In addition, in a case where a difference between an angle θ (see FIG. 12) between the current posture (extension direction) of the puncture needle 18 and the extension direction of the recommended puncture route 72 becomes equal to or greater than a predetermined angle threshold, the puncture needle deviation determination unit 64 determines that the puncture needle 18 has deviated from the recommended puncture route 72.

In the present embodiment, the puncture needle deviation determination unit 64 calculates the distance between the puncture needle 18 at the current position and the recommended puncture route 72 or the difference in angle from the extension direction in the model coordinate system, but the puncture needle deviation determination unit 64 may perform the calculation in the real space coordinate system.

In addition, as shown in FIG. 11, in a case where the cross section of the real-time image 84 and the cross section of the reconstructed ultrasound image 80 are the same cross section, the puncture needle deviation determination unit 64 may determine that the puncture needle 18 has deviated from the recommended puncture route 72 in consideration of the comparison result between an image 90 of the puncture needle 18 included in the real-time image 84 and the recommended puncture route image 82.

The display control unit 36 as the notification processing unit notifies the operator in a case where the puncture needle deviation determination unit 64 determines that the puncture needle 18 has deviated from the recommended puncture route 72.

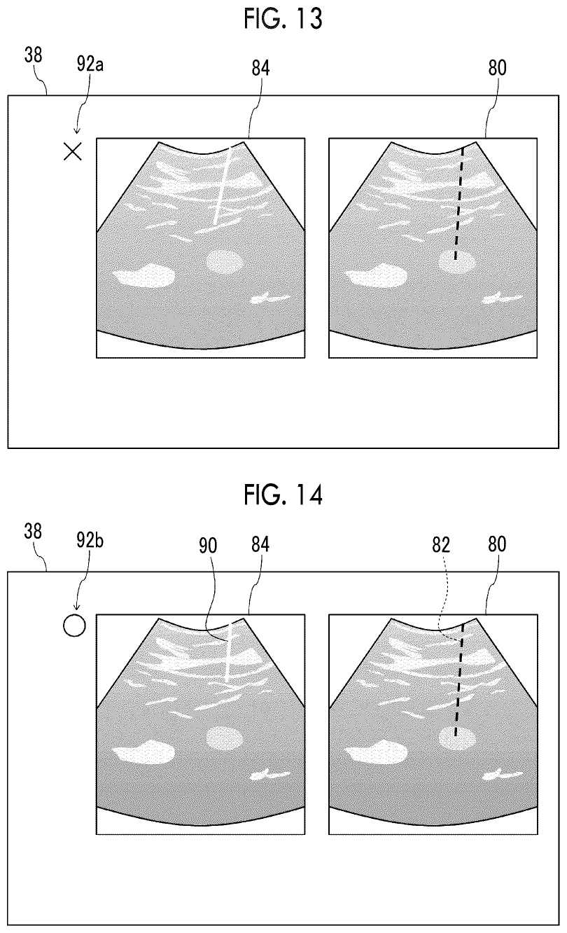

FIG. 13 is a diagram showing an example of notification that the puncture needle 18 has deviated. In a case where it is determined that the puncture needle 18 has deviated from the recommended puncture route 72, the display control unit 36 notifies the operator that the puncture needle 18 has deviated from the recommended puncture route 72 by displaying, for example, an icon 92a as shown in FIG. 13. The display control unit 36 may change the icon 92a to be displayed according to the amount of deviation of the puncture needle 18 from the recommended puncture route 72. For example, the display control unit 36 may display a Δ mark as the icon 92a in a case where the puncture needle 18 has deviated from the recommended puncture route 72 but the amount of deviation is relatively small, and may display a x mark as the icon 92a in a case where the amount of deviation is relatively large.

FIG. 14 is a diagram showing an example of notification that the puncture needle 18 has not deviated. Even in a case where the puncture needle 18 has not deviated from the recommended puncture route 72, the display control unit 36 may notify the operator that the puncture needle 18 has not deviated from the recommended puncture route 72 by displaying, for example, an icon 92b different from the icon 92a as shown in FIG. 14.

Although the puncture support apparatus according to the present disclosure has been described above, the puncture support apparatus according to the present disclosure is not limited to the above-described embodiment, and various changes can be made without departing from the gist thereof.

For example, in each of the above-described embodiments, the puncture support apparatus is the ultrasound diagnostic apparatus 16, and each of the functions of the image forming unit 34, the display control unit 36, the probe information acquisition unit 50, the volume data acquisition unit 52, the model forming unit 54, the puncture route specifying unit 56, the recommended position and posture specifying unit 58, the consistency index calculation unit 60, the puncture needle information acquisition unit 62, and the puncture needle deviation determination unit 64 is included in the ultrasound diagnostic apparatus 16. However, each of these functions does not necessarily have to be exhibited by the ultrasound diagnostic apparatus 16. For example, these functions may be exhibited by a server computer or the like that is communicatively connected to the ultrasound diagnostic apparatus 16. In addition, all of the above-described functions may not be exhibited by one device, and the above-described functions may be exhibited by cooperation of a plurality of devices.

What is claimed is:

1. A puncture support apparatus comprising:
a probe information acquisition unit that acquires probe information indicating a position and a posture of an ultrasound probe;
a volume data acquisition unit that acquires volume data based on a reception signal obtained by transmitting and receiving ultrasound from the ultrasound probe toward a subject;
a model forming unit that forms a subject model including a puncture target object model that is a three-dimensional model representing a puncture target object, and an avoidance tissue model that is a three-dimensional model representing an avoidance tissue which is a tissue to be avoided by a puncture needle while puncturing the puncture target object, based on the volume data, the subject model having position information indicating each position of the subject model based on the position and the posture of the ultrasound probe in a case where the reception signal is acquired;
a puncture route specifying unit that specifies a recommended puncture route which is a route that extends from a body surface position of the subject to the puncture target object model while avoiding the avoidance tissue model in the subject model and through which the puncture needle is to pass; and
a notification processing unit that notifies an operator of the recommended puncture route.

2. The puncture support apparatus according to claim 1,
wherein the puncture route specifying unit specifies the recommended puncture route such that a shortest distance between the recommended puncture route and the avoidance tissue model is equal to or greater than a first threshold distance.

3. The puncture support apparatus according to claim 1,
wherein, in a case where the puncture route specifying unit specifies a plurality of candidate puncture routes that extend from the body surface position of the subject to the puncture target object model while avoiding the avoidance tissue model, the puncture route specifying unit specifies, as the recommended puncture route, a candidate puncture route having a shortest length among the plurality of candidate puncture routes.

4. The puncture support apparatus according to claim 1,
wherein the puncture target object is a tumor,
the puncture needle is an ablation puncture needle for ablating the tumor, and
the puncture route specifying unit specifies the recommended puncture route such that an end part of the recommended puncture route on a tumor model side as the puncture target object model is at a second threshold distance or more from a tissue model other than a tumor model.

5. The puncture support apparatus according to claim 1, further comprising:
a recommended position and posture specifying unit that specifies a recommended probe position and posture that corresponds to the position and the posture of the ultrasound probe such that a scanning plane of the ultrasound probe includes the recommended puncture route and is parallel to the recommended puncture route, based on the recommended puncture route,
wherein the notification processing unit notifies the operator of the recommended probe position and posture.

6. The puncture support apparatus according to claim 5,
wherein the notification processing unit notifies the operator of guide information for transitioning the position and the posture of the ultrasound probe from a current position and a current posture of the ultrasound probe to the recommended probe position and posture based on a difference between the current position and the current posture of the ultrasound probe and the recommended probe position and posture.

7. The puncture support apparatus according to claim 6, further comprising:
a consistency index calculation unit that calculates a consistency index that is an index indicating a degree of matching between a reconstructed ultrasound image cut out from the subject model in a cross section that includes the recommended puncture route and is parallel to the recommended puncture route, and a real-time image that is an ultrasound tomographic image formed at the current position and the current posture of the ultrasound probe,
wherein the notification processing unit notifies the operator of the consistency index.

8. The puncture support apparatus according to claim 1, further comprising:
a puncture needle information acquisition unit that acquires puncture needle information indicating a current position and a current posture of the puncture needle; and
a puncture needle deviation determination unit that determines that the puncture needle has deviated from the recommended puncture route in a case where a distance between the puncture needle at the current position and the recommended puncture route becomes equal to or greater than a distance threshold or in a case where a difference in angle between a current extension direction of the puncture needle and an extension direction of the recommended puncture route becomes equal to or greater than an angle threshold,
wherein the notification processing unit notifies the operator in a case where it is determined that the puncture needle has deviated from the recommended puncture route.

9. The puncture support apparatus according to claim 1,
wherein the probe information acquisition unit detects a position and a posture of the ultrasound probe relative to a body surface of the subject based on a captured image acquired by imaging a probe detection marker attached to the ultrasound probe and a body surface detection marker attached to the body surface of the subject via a camera.

10. The puncture support apparatus according to claim 8,
wherein the probe information acquisition unit detects a position and a posture of the ultrasound probe relative to a body surface of the subject based on a captured image acquired by imaging a probe detection marker attached to the ultrasound probe and a body surface detection marker attached to the body surface of the subject via a camera, and
the puncture needle information acquisition unit detects a position and a posture of the puncture needle relative to the body surface of the subject based on a captured image acquired by imaging a puncture needle detection marker attached to the puncture needle and the body surface detection marker via the camera.

11. A non-transitory computer-readable storage medium storing a puncture support program causing a computer to function as:
a probe information acquisition unit that acquires probe information indicating a position and a posture of an ultrasound probe;
a volume data acquisition unit that acquires volume data based on a reception signal obtained by transmitting and receiving ultrasound from the ultrasound probe toward a subject;
a model forming unit that forms a subject model including a puncture target object model that is a three-dimensional model representing a puncture target object, and an avoidance tissue model that is a three-dimensional model representing an avoidance tissue which is a tissue to be avoided by a puncture needle while puncturing the puncture target object, based on the volume data, the subject model having position information indicating each position of the subject model based on the position and the posture of the ultrasound probe in a case where the reception signal is acquired; and
a puncture route specifying unit that specifies a recommended puncture route which is a route that extends from a body surface position of the subject to the puncture target object model while avoiding the avoidance tissue model in the subject model and through which the puncture needle is to pass.
